(19) United States
(12) Patent Application Publication
(10) Pub. No.: US 2016/0185697 A1
(43) Pub. Date: Jun. 30, 2016

(54) METHOD FOR PREPARING ISOPRENOL-ALKOXYLATE COMPOSITIONS HAVING A LOW ISOPRENE-CONTENT

(71) Applicant: BASF SE, Ludwigshafen (DE)

(72) Inventors: Martin ERNST, Heidelberg (DE); Sebastian HASSELBACH, Grünstadt (DE); Stefan MÜSSIG, Ellerstadt (DE)

(21) Appl. No.: 14/911,575

(22) PCT Filed: Jul. 29, 2014
(86) PCT No.: PCT/EP2014/066268

§ 371 Date: Feb. 11, 2016

(30) Foreign Application Priority Data
Aug. 15, 2013 (EP) 13180556.6

(51) Int. Cl. C07C 41/03; C07C 43/178

(52) U.S. Cl. CPC C07C 41/03; C07C 43/178

(57) ABSTRACT

The present invention relates to methods for preparing a composition comprising an isoprenol-alkoxylate having an isoprene-content of not more than 1000 ppm. The present invention also relates to compositions prepared or obtainable by such methods. The present invention further relates to the use of peroxides for decreasing the isoprene-content in a composition.

The present invention relates to methods for preparing a composition comprising an isoprenol-alkoxylate having an isoprene-content of not more than 1000 ppm. The present invention also relates to compositions prepared or obtainable by such methods. The present invention further relates to the use of peroxides for decreasing the isoprene-content in a composition.

Isoprenol-alkoxylates are important raw materials for the downstream chemical industry, e.g., the production of superplasticizers for concrete (see, e.g., EP2090596 A1 or WO2002096823 A1). Typical isoprenol-alkoxylates are prepared from isoprenol together with ethylene oxide (EO) and optionally propylene oxide (PO) (see, e.g., CN 102140167 A, CN 101928392 A, JP 2012057093). In this process, a certain amount of isoprene is synthesized as a by-product (see, e.g., WO 08/126909 and EP-B1 1213315). Without being bound by theory, it is believed that isoprenol or isoprenol-alkoxylate is decomposed under alkaline conditions (the same holds true for acidic conditions which are, however, not usual in alkoxylation) thereby forming isoprene. Common methods for decreasing the isoprene-content in a composition are physical approaches such as evacuation by vacuum (see, e.g., EP-A1 066179) of the composition at the end of the alkoxylation process in order to remove residual oxide, and/or to further reduce the isoprene-content by stripping (e.g., adding inert gas and/or water (steam)) (see, e.g., EP-B1 965605 or EP-A1 2333002). Also, the influence of UV light on polymerization of isoprene has been analyzed (Elkanzi, J Hazardous Materials (2000), 73(1): 55-62).

Isoprene is a CMR compound (carcinogenic according to GHS category 1B, and mutagenic according to GHS category 2; cf. REACh registration no. 01-2119457891-29-0000; EC-no. 201-143-3). Although there is no obligation for labeling isoprene-containing compositions comprising isoprene at a content of <1,000 ppm, there are certainly residual environmental and health risks when handling such compositions which can only be minimized or even erased with a virtual complete deletion or removal of isoprene in the composition. However, physical methods for decreasing the isoprene-content in an isoprenol-alkoxylate composition bear the disadvantage that isoprene is still contained in the exhaust gas which may still have the potential to harm the environment.

Thus, there is a need for a chemical method of preparing an isoprenol-alkoxylate composition having a low isoprene content.

This technical problem has been solved by the present invention as set forth in the claims and as described and exemplified herein.

The present invention is based on the surprising finding that the isoprene-content can be drastically reduced in the preparation of isoprenol-alkoxylate compositions by adding a peroxide or peroxide generating compound to the reaction mixture. Without being bound by theory, the peroxide or peroxide generating compound may generate a cyclic peroxide from one of the double bonds and decomposes to low molecular weight alcohols and aldehydes. This chemical isoprene depletion method according to the present invention is completely independent from further methods of reducing the isoprene content, e.g., physical removal and/or polymerization of isoprene under UV radiation influence as known in the art. Thus, the method of the present invention bears the advantage that it is more reliable and more adjustable than the physical removal methods or UV radiation-induced polymerization methods of the prior art. Furthermore, the inventive method bears lower risks for the environment as by using methods of physical removal of isoprene, isoprene may still be contained in the exhaust gas.

Therefore, in one embodiment, the method of the present invention as further described and provided herein is carried out without further physical removal of isoprene (e.g., evacuation or stripping with inert gas, water and/or water steam in order to remove oxides as further described herein below) and/or without employing artificial UV radiation to polymerize isoprene. In this context, as also further described herein, the term “artificial UV radiation” is to be understood as UV radiation treatment of the reaction mixture containing isoprene which exceeds UV radiation levels of usual environmental day light and/or UV radiation emitted from common electric illumination. In essence, in this embodiment, the composition to be prepared by the method of the present invention is not treated with UV radiation which is capable of passing through common glass, acrylic glass or quartz glass panels. For example, the composition to be prepared by the method of the present invention is not treated with UV radiation having a wavelength of 400 nm or less (does not pass common glass panels), preferably 300 nm or less, and more preferably 200 nm or less (does not pass common acrylic glass or quartz glass panels) and an irradiance intensity of above 500 W/m² as measured with an irradiance meter. In this context, “treatment with UV radiation” means treatment which is sufficient to polymerize (and, thus, eliminate) substantial amounts isoprene from the treated composition, e.g., more than 5%, 10%, 20% or 25% of the isoprene contained in the composition before UV radiation treatment. Generally. UV radiation can be measured as known in the art, preferably by the method described in Diffey, Methods (2002), 28: 4-13.

In addition, as a side-effect, the isoprenol-alkoxylate prepared by the method of the present invention has a lighter color compared to a product in which the isoprene-content was not reduced accordingly. Thus, the present invention also allows adjustment of color of the produced isoprenol-alkoxylate while reducing the content of the unwanted by-product isoprene.

The present invention relates to a method for preparing a composition comprising an isoprenol-alkoxylate having an isoprene-content of not more than 1000, preferably not more than 500, more preferably not more than 100, more preferably not more than 50, more preferably not more than 10 ppm, and most preferably not more than 1 ppm, said method comprising the following steps:
(a) reacting isoprenol with at least one alkylene oxide and a catalyst at a temperature between 50° C. and 200° C.;
(b) optionally eliminating residual oxide;
(c) optionally adding an inert gas or adding and subsequently removing water or water steam;
(d) adding a peroxide or peroxide generating compound to the mixture resulting from (a) to (c); and
(e) homogenizing the mixture resulting from (d).

In context with the present invention, during step (a) of the inventive method, the at least one alkylene oxide to be reacted with isoprenol may be any suitable alkylene oxide. Typical examples for the alkylene oxide which may be employed in this context include ethylene oxide (EO), propylene oxide (PO), butylene oxide (BuO), pentene oxide (PentenO), decene oxide (DecenO), and dodecene oxide (DodecenO). In one embodiment, the alkylene oxide to be reacted with isoprenol is EO or PO.

The catalyst to be employed in step (a) of the method according to the present invention may be any catalyst capable of allowing reaction of isoprenol and an alkylene oxide to an isoprenol-alkoxylate. Examples for such catalysts include BF3, alkaline alkoxylates (e.g., Na-alkoxylate, alkoxylate or K-alkoxylate, KOMe) or -hydroxides (e.g., NaOH or KOH), double-metal cyanides, tertiary amines, triphenylphosphine, NaH, Na, KH, and carboxylic salts. In one embodiment, the catalyst to be employed in the method of the present invention is KOMe.

The reaction temperature in step (a) of the method of the present invention generally lies between 50° C. and 200° C., preferably between 70° C. to 180° C., more preferably between 80° C. to 170° C., more preferably 90° C. to 160° C., and most preferably 100° C. to 150° C. For example, the pressure may be 1 to 20 bar, preferably 1 to 10 bar, and most preferably 1 to 6 bar.

According to the present invention, after reaction step (a) of the method provided herein, it is possible to apply a step (b) of removing or eliminating residual oxide by different means, e.g., by evacuation via vacuum as known in the art.

Another optional step (c) of decreasing the isoprene content in advance is stripping with inert gas and/or water (steam) as known in the art and as further described and exemplified herein. For example, it is possible to add an inert gas (e.g., N₂) or to add and subsequently remove water (steam) to the reaction mixture resulting from step (a) of the method described and provided herein. In this way, some easily desorbed isoprene can be removed and optionally burned in the flare while the subsequent chemical depletion method of this invention can be conducted more safely utilizing smaller amounts of, e.g., peracetic acid or hydrogen peroxide.

Furthermore, as a subsequent step (d) of the method of the present invention, a peroxide and/or a peroxide generating compound is added to the reaction mixture (optionally treated with inert gas and/or water (steam) as described above). As described and exemplified herein, in context with the present invention it has surprisingly been found that peroxides (or peroxide generating compounds) are capable of drastically reducing the content of isoprene in isoprenol-alkoxylate preparations. This effect is independent from other isoprene-removal methods such as physical methods or UV radiation. In context with the present invention, suitable peroxides or peroxide generating compounds comprise inter alia peracetic acid and salts thereof, hydrogen peroxide and salts thereof, Na₂O₂, K₂O₂, and other alkaline earth metal or peroxide salts like, e.g., sodium perborate. Preferred peroxides are peracetic acid and hydrogen peroxide.

In context with the present invention, the peroxides or peroxide generating compounds may be added as aqueous solution. Such aqueous solutions may contain, for example, 1% to 95%, preferably 10% to 80%, more preferably 20% to 70%, and most preferably 30% to 50% peroxide or peroxide generating compound.

After adding peroxide or peroxide generating compound in step (d) of the method described and provided herein, the resulting mixture may contain 1 to 10,000 ppm, preferably 10 to 10,000 ppm, more preferably 50 to 5,000 ppm, more preferably 100 to 1,000 ppm, and most preferably 300 to 1,000 ppm peroxide at to (i.e. directly) after addition of the peroxide/peroxide generating compound. In this context, the term “t₀ after addition of the peroxide/peroxide generating compound” is to be construed as the quickest time point possible to take a sample of the resulting mixture after addition of the peroxide/peroxide generating compound to measure the peroxide content of the resulting mixture. For example, it is practically not possible to measure the peroxide content of the resulting mixture instantly (i.e. without any time loss) after addition of a peroxide/peroxide generating compound. That is, in context with the method described and provided herein, the term “to after addition of the peroxide/peroxide generating compound” includes the period of time necessary for the skilled person to take a sample of the resulting mixture after addition of a peroxide/peroxide generating compound. Such period of time may be, e.g., up to 10 to 20 minutes after addition of the peroxide/peroxide generating compound, bearing in mind that the solution should be homogenized. Methods for measuring the peroxide content in a composition are well known in the art and comprise, inter alia, the reaction of iodide with peroxides (Lea, Proc Royal Soc, (1931), 108: 175-189) or titration of hydroperoxides with lithium aluminium hydride (Higuchi, J Am Chem Soc (1951), 73: 2676-2679).

Finally, the method of the present invention comprises a step (e) of homogenizing the mixture obtained after adding a peroxide and/or a peroxide generating compound in step (d). This homogenizing step (e) may be performed at any suitable temperature, particularly at temperatures between 0° C. to 160° C., preferably between 15° C. to 150° C., more preferably between 20° C. to 140° C., more preferably between 30° C. to 130° C., more preferably between 40° C. to 120° C., more preferably between 50° C. to 120° C., and most preferably between 60° C. to 120° C. “Homogenizing” in context with the present invention does not necessarily mean that 100% of the mixture must be completely homogenized. “Homogenizing” in this context means that the mixture is largely homogenized to a degree technically possible for the skilled person and as usual-ly reached when applying common homogenizing methods known in the art. Such homogenizing methods include, e.g., stirring with a mechanical stirrer, recirculation by pumping in conjunc-tion with a static mixer, or convection using gas bubbles as known in the art. This step may be performed for a time sufficient to reach the desired isoprene content. As described and exemplified herein, the isoprene content is decreasing after addition of peroxide or peroxide generating compound in step (d) over the time. For example, the homogenizing step (e) according to the present invention (e.g., by stirring) may be performed for 1 to 300 min, preferably for 10 to 300 min, more preferably for 10 to 240 min, more preferably for 30 to 240 min, and most preferably for 30 to 180 min.

In context with the present invention, the pH of the compositions to be prepared by the method described and provided herein may be adjusted to 2 to 12, more preferably to 4 to 12, and most preferably to 5 to 11 as measured in 10% aqueous solution corresponding to DIN 19268.

In one aspect of the present invention, the compositions to be prepared by the method provided herein or the isoprenol-alkoxylates contained therein are not polymerized during the treatment. In a further aspect of the present invention, the compositions to be prepared by the method provided herein or the isoprenol-alkoxylates contained therein are not polymerized after the preparation method as described and provided herein at all.

Accordingly, in one embodiment of the present invention, the isoprenol-alkoxylate composition to be prepared by the method described and provided herein is free of polymerization catalysts selected from the group consisting of Sc, Ti, V, Cr, Mn, Fe, Co Ni, Cu and Zn, wherein the degree of oxidation of the catalyst is not relevant. In this context, such catalysts may also comprise the respective ions in coordinated form, e.g., in coordination with EDTA or Trilon®. In this context, the term “free of polymerization catalysts” means that no such catalysts are added as such to the reaction mixture before or after the homogenizing step (e) of the method provided herein. Also, the term “free of polymerization catalysts” means that the composition is essentially free of such catalysts, i.e. it does not necessarily mean that no single atom of such catalysts may be present in the composition to be prepared but small amounts which can be considered as impu-rities may be allowed. For example, the term “free of polymerization catalysts” may mean that up to 100 ppm, preferably up to 50 ppm, and most preferably up to 10 ppm of such catalysts may be present in the composition to be prepared by the method of the present invention. Methods for measuring the concentration of such catalysts are known in the art and comprise, e.g., titration and atomic adsorption spectrometry (see, e.g., Welz. Atomabsorptionsspek-trometrie, 4^{th} edition (Weinheim 1999), ISBN 3-527-28305-6; Skoog, Instrumentelle Analytik (Berlin 1996), ISBN 3-540-60450-2; Wünsch, Optische Analyseverfahren zur Bestimmung anor-ganischer Stoffe, Sammlung Göschen, Bd. 2606, de Gruyter Berlin, ISBN 3-11-003908-7).

Furthermore, the isoprenol-alkoxylate composition to be prepared by the method described and provided herein may be free of unsaturated copolymerizable acids. In this context, the term “free of unsaturated copolymerizable acids” also comprises compositions which are essentially free of such acids, i.e. said term does not necessarily mean that no molecule of such acids must be contained in the isoprenol-alkoxylate composition. For example, the term “free of unsaturated copolymerizable acids” may mean that up to 2.0 wt %, preferably up to 1.5 wt % more preferably up to 1.0 wt %, and most preferably up to 0.5 wt % of unsaturated copolymerizable acids may be contained in the isoprenol-alkoxylate composition to be prepared by the method described and provided herein. In this context, examples of such unsaturated copolymerizable acids comprise acrylic acid, methacrylic acid, maleic acid, and itaconic acid. The content of such acids may be measured by methods known in the art, e.g., by acid-base titration (preferred). HPLC, or vapor phase chromatography.

Furthermore, in one embodiment of the invention, the isoprenol-alkoxylate composition to be prepared by the method described and provided herein may be free of isoprenyl-alkoxylate homo- and copolymers. In this context, the term “free of isoprenyl-alkoxylate homo- and copolymers”also comprises compositions which are essentially free of such homo- or copolymers, i.e. said term does not necessarily mean that no molecule of such homo- or copolymers must be contained in the isoprenol-alkoxylate composition. For example, the term “free of unsaturated copolymerizable acids” may mean that up to 1000 ppm may be contained in the composition to be prepared by the method of the present invention. The amount of such homo- or copolymers may be measured by methods known in the art, e.g., Gel permeation chromatography or HPLC.

In one embodiment, the method of the present invention as further described and provided herein is carried out without further physical removal of isoprene (e.g., evacuation or stripping with inert gas, water and/or water steam in order to remove oxides as further described herein above) and/or without employing artificial UV radiation to polymerize isoprene. In this context, as also further described herein, the term “artificial UV radiation” is to be understood as UV radiation treatment of the reaction mixture containing isoprene which exceeds UV radiation levels of usual environmental day light and/or UV radiation emitted from common electric illumination. In essence, in this embodiment, the composition to be prepared by the method of the present invention is not treated with UV radiation which is capable of passing through common glass, acrylic glass or quartz glass panels. For example, the composition to be prepared by the method of the present invention is not treated with UV radiation having a wavelength of 400 nm or less (does not pass common glass panels), preferably 300 nm or less, and more preferably 200 nm or less (does not pass common acrylic glass or quartz glass panels) and an irradiance intensity of above 500 W/m² as measured with an irradiance meter. In this context, “treatment with UV radiation” means treatment which is sufficient to polymerize (and, thus, eliminate) substantial amounts isoprene from the treated composition, e.g., more than 5%, 10%, 20% or 25% of the isoprene contained in the composition before UV radiation treatment. UV radiation can be measured by methods known in the art, preferably by the method described in Diffey, Methods (2002), 28: 4-13.

As already described above, the method of the present invention also allows the preparation of lighter products, i.e. isoprenol-alkoxylate compositions having a reduced color number corn-pared to the compositions before addition of a peroxide or peroxide generating compound as described herein. Accordingly, in one embodiment, the present invention also relates to a method for preparing an isoprenol-alkoxylate containing composition as described and exemplified herein, wherein the color number (Gardner) is reduced from over 5.5 (before addition of peroxide/peroxide generating compound as described herein) to below 5 (after addition of peroxide/peroxide generating compound as described herein)), preferably from over 5.5 to below 4.8, more preferably from over 5.5 to below 4.5, and most preferably from over 5 to below 4.5. The color can be measured by, e.g., using a spectral photometer produced by Hach Lange GmbH according to EN 1557 and as also exemplified herein below.

Generally, the present invention also relates to compositions prepared by the method of the present invention as described and provided herein.

The present invention also relates to the use of peroxides or peroxide generating compounds as described hereinabove for decreasing the amount of isoprene in a composition. For example, in this context, the isoprene amount may be decreased by 10% to 99.99%, more preferably 50 to 99.99%, most preferably 90 to 99.99% by adding a peroxide and/or peroxide generating compound as described hereinabove. In particular, the present invention relates to the corresponding use of such peroxides or peroxide generating compounds for decreasing the amount of isoprene in isoprenol-alkoxylate containing compositions.

Without being bound by theory, prenol is an isomer of isoprenol which might isomerize to some extent and release traces of isoprene. Thus, in context with the present invention, when refer-ring to isoprene-contents or -amounts in compositions to be prepared by the method of the present invention, also prenol-containing compositions shall generally be comprised by terms such as “isoprenol-alkoxylate containing composition” or “isoprenol-alkoxylate composition”. Accordingly, the method provided and described herein is also useful for preparing prenol-alkoxylate containing compositions having a low isoprene-content as described hereinabove. Analogously, the present invention also relates to the use of peroxides or peroxide generating compounds for decreasing the amount of isoprene in prenol-alkoxylate containing compositions as described hereinabove.

The following examples illustrate the present invention. However, the examples must not be construed as limiting the present invention to the features and embodiments detailed in the examples.

### EXAMPLES

### Example 1

### Description of Analytical Method for Measuring Isoprene Content

The isoprene content was determined as follows. A capillary gas chromatograph with FID and Headspace sampling unit (Perkin Elmer Clarus 600 with Turbomatrix 110) with column CP-Wax 52 CB 30×0.32 mm×0.5 μm was used. Reagents used were isoprene for analysis and N,N-Dimethylacetamide (DMAA) for analysis obtained from Aldrich. Vials and vial caps were obtained from Ziemer.

The sample was analyzed on a CP-Wax stationary phase by gas chromatography. The content was determined by standard addition method. The sensitivity range was 1 to 100 ppm.

Parameters on the autosampler were set as follows:

Temperature of sample: 70° C.

Temperature of needle: 90° C.

Temperature of transfer tube: 90° C.

Thermostatising time: 45.00 min

Pressurization time: 1.0 min

Injection time: 0.1 min

Needle residence time: 0.20 min

Vent time: 0.10 min

Cycle time: 54.00 min

Parameters on the 9 as chromatograph were set as follows:

Temp. 1: 50° C.

Time 1: 5 min.

Rate: 10° C./min.

Temp. 2: 240° C.

Time 2: 16 min

Detector temp.: 220° C.

Injector temp.: 150° C.

Pre-Column pressure: 9 psi

Carrier gas: Helium

Preparation of Calibration Solutions:

Standard stock solution: weigh approx. 250 mg Isoprene for analysis into a 25 mL graded measuring cylinder with exactitude of 0.0001 g and complete with DMAA to the mark.

Calibration Solution 1: add 100 μl stock solution into a 100 mL graded measuring cylinder and complete with DMAA to the mark. The final solution then contains approx. 10 ppm of isoprene.

Calibration Solution 2: add 500 μl stock solution into a 100 mL graded measuring cylinder and complete with DMAA to the mark. The final solution then contains approx. 50 ppm of isoprene.

The solutions are tightly stoppered and have a shelf life of about 3 months if stored in the refrigerator.

Sample Preparation:

Sample vial 0: 1 g sample+1 g DMAA for analysis

Sample vial 1: 1 g sample+1 g calibration solution 1 (addition 1)

Sample vial 2: 1 g sample+1 g calibration solution 2 (addition 2)

Samples with high isoprene content have to be diluted correspondingly with DMAA or less sample is weighed in.

Calculation:

$$W 1 = \frac{E \times peak area . Add .0}{( peak area Add .1 - p eak area Add .0 )}$$

W1=weight fraction in ppm isoprene

E=concentration of calibration solution in ppm (Isoprene)

Peak area. Add. 0=peak area of sample (Vial 0+Sample+DMAA)

Peak area. Add. 1=peak area of sample+peak area of calibration solution 1

$$W 2 = \frac{E \times peak area . Add .0}{( peak area Add . 2 - p eak area Add .0 )}$$

W2=weight fraction in ppm isoprene

E=concentration of calibration solution in ppm (Isoprene)

Peak area. Add. 0=peak area of sample (Vial 0: +Sample+DMAA)

Peak area. Add. 2=peak area of sample+peak area of calibration solution 1

For the calculation peak areas are set to 1 g. From mass fraction W1 and W2 the average is calculated.

### Example 2

### Measurement of Color Number with Colorimeter Lica 200 by Hach Lange GmbH According to EN 1557

The apparatus was switched on and left to warm up for 15 minutes. Prior to use it was calibrated with distilled water. A cylindrical cuvette with 11 mm diameter was filled with distilled water to 75% of capacity and put into the apparatus. The cuvette was completely clean. Fingerprints on the cuvette and air bubbles adhering to the glass were avoided. The lid of the apparatus was closed and the calibration was started. After calibration was confirmed, the cuvette containing distilled water was removed. Another cylindrical cuvette was filled with the sample to 75% of capacity and put into the apparatus. After having taken the precautions described above, the measurement was started. The values for color in Gardner (and Hazen and iodine) units were displayed and printed out. Liquid samples were analyzed at room temperature, solid samples were melted at 80° C. and once completely homogeneous, immediately analyzed.

### Example 3

### Reduction of Isoprene-Content in Isoprenol-Alkoxylate Containing Composition 1

10 g of an isoprenol alkoxylate prepared by reacting 1 mol isoprenol with 2 mol ethylene oxide (reaction temperature 130° C.) and 2 mol propylene oxide (reaction temperature 125° C.) using potassium methylate as catalyst and neutralized with the appropriate amount of acetic acid were heated in a heating chamber to 75° C. After having reached the set temperature, 0.1 g 30% H₂O₂ was added to the mixture. Subsequently, the weighing recipient containing the H₂O₂ was rinsed with app. 0.1 g water and which was also added to the mixture. The mixture was stirred on a heating plate with magnetic stirrer at 50° C. After app. 80 min at 50° C., the reaction mixture was analyzed for its isoprene content as described in Example 1. Also, the color number (Gardner, iodine) was measured before and after addition of H₂O₂ as described above.

TABLE 1
Isoprene-content of Composition 1

| | Isoprene | Color number | Color number |
|---|---|---|---|
| Composition/Mixture | content | (Gardner) | (iodine) |
| Isoprenol + 2EO + 2PO | 320 ppm | 5.7 | 7.6 |
| Isoprenol + 2EO + 2PO + | <1 ppm | 4.4 | 4.7 |
| 1% H₂O₂ (w = 30%) | | | |

### Example 4

### Reduction of Isoprene-Content in Isoprenol-Alkoxylate Containing Compositions 2-7

The amount of isoprenol alkoxylate (Isoprenyl-polyethylene glykol of Mw 1100 preparation analogous to the description provided herein above, reaction temperature approximately 130° C.-140° C.) as specified in Table 2 was charged into a 1 l four-neck flask equipped with a mechanical stirrer from above. After heating to the specified reaction temperature, the specified amount of H₂O₂ was added and the mixture was stirred for the time given at 225 rpm.

TABLE 2
Isoprene-content of Compositions 2 to 7

| Composition | Starting | Amount | Amount | Reaction | isoprene before | isoprene | isoprene | isoprene | Color | Color |
|---|---|---|---|---|---|---|---|---|---|---|
| no. | Material | [g] | Reactant | Conditions | treatment | 1 hr | 2 hr | 3 hr | before* | after* |
| 2 | Isoprenyl- | 924.03 | 600 ppm | 80° C., | 240 ppm | 90 ppm | 30 ppm | 8 ppm | 5.3 | 2.7 |
| | polyethylene | | H₂O₂ | addition | | | | | | |
| | glykol of | | | H₂O₂ over | | | | | | |
| | Mw 1100 | | | 10 min | | | | | | |
| 3 | Isoprenyl- | 975.32 | 600 ppm | 100° C., | 210 ppm | 20 ppm | 5 ppm | 1 ppm | 5.4 | 2.9 |
| | polyethylene | | H₂O₂ | addition | | | | | | |
| | glykol of | | | H₂O₂ over | | | | | | |
| | Mw 1100 | | | 10 min | | | | | | |
| 4 | Isoprenyl- | 944.86 | 600 ppm | 110° C., | 190 ppm | 40 ppm | 8 ppm | 2 ppm | 3.1 | 1.5 |
| | polyethylene | | H₂O₂ | addition | | | | | | |
| | glykol of | | | H₂O₂ over | | | | | | |
| | Mw 1100 | | | 10 min | | | | | | |
| 5 | Isoprenyl- | 945.36 | 600 ppm | 120° C., | 200 ppm | 15 ppm | 1 ppm | <1 ppm | 4.9 | 2.2 |
| | polyethylene | | H₂O₂ | addition | | | | | | |
| | glykol of | | | H₂O₂ over | | | | | | |
| | Mw 1100 | | | 10 min at | | | | | | |
| | | | | 100° C. | | | | | | |
| 6 | Isoprenyl- | 984.83 | 600 ppm | 100° C., | 98 ppm | 23 ppm | 5 ppm | <1 ppm | 4.6 | 2.7 |
| | polyethylene | | H₂O₂ | addition | | | | | | |
| | glykol of | | | H₂O₂ over | | | | | | |
| | Mw 1100 | | | 10 min, | | | | | | |
| | | | | in the dark** | | | | | | |
| 7 | Isoprenol + | 852.8 | 1,200 ppm | 80° C., | 83 ppm | 44 ppm | <1 ppm | <1 ppm | 4.1 | 3.2 |
| | 2EO + 2PO | | peracetic | addition | | | | | | |
| | | | acid | peracetic | | | | | | |
| | | | | acid over | | | | | | |
| | | | | 15 min | | | | | | |

*Color after Gardner
**,,in the dark” means that the flask was wrapped in aluminum foil and the hood was kept without artificial illumination

### Example 5

### Treatment of Isoprenol Alkoxylate with N₂

Isoprenol was alkoxylated in an 1 l pressure autoclave with 2 moles EO and 2 moles PO using 3400 ppm KOMe as catalyst. When the drop in pressure indicated complete conversion, a sample was taken and the isoprene content measured as described above (870 ppm). The remaining product (700 g) was treated by bubbling through N₂ with a rate of 7.5 l/h at 125° C. The isoprene content was measured again and found to be 110 pm. 500 g of the product were then treated in a flask with N₂ at a rate of 7 k N₂/h at 125° C. and after another 1 and 2 h the isoprene content was analyzed and found to be 65 and 54 ppm, respectively.

That is, after treatment of isoprenyl alkoxylate for 3 h with N₂, the isoprene content was >50 ppm.

1.-20. (canceled)

21. A method for preparing a composition comprising an isoprenol-alkoxylate having an isoprene-content of not more than 1000 said method comprising the following steps:
(a) reacting isoprenol with at least one alkylene oxide and a catalyst at a temperature between 50° C. and 200° C.;
(b) optionally eliminating residual oxide;
(c) optionally adding an inert gas or adding and subsequently removing water or water steam;
(d) adding a peroxide or peroxide generating compound to the mixture resulting from (a) to (c); and
(e) homogenizing the mixture resulting from (d).

22. The method of claim 21, wherein the homogenizing step (e) is performed at a temperature of 0° C. to 160° C.

23. The method of claim 21, wherein said peroxide or peroxide generating compound in step (d) is selected from the group consisting of peracetic acid and salts thereof, hydrogen peroxide and salts thereof, Na₂O₂, K₂O₂, perborates, and other alkaline earth metal or peroxide salts.

24. The method of claim 21, wherein said peroxide is added as aqueous solution.

25. The method of claim 24, wherein said aqueous peroxide solution contains 1 to 95% peroxide.

26. The method of claim 21, wherein said mixture resulting from (d) contains 1 to 10,000 ppm peroxide at t₀ after addition of the peroxide.

27. The method of claim 21, wherein the pH of said composition comprising an isoprenol-alkoxylate is adjusted to 2 to 12 as measured in 10% aqueous solution.

28. The method of claim 21, wherein said composition is free or essentially free of polymerization catalysts selected from the group consisting of Sc, Ti, V, Cr, Mn, Fe, Co, Ni, Cu, and Zn.

29. The method of claim 21, wherein said composition is free or essentially free of unsaturated copolymerizable acids.

30. The method of claim 29, wherein said unsaturated copolymerizable acid is selected from the group consisting of acrylic acid, methacrylic acid, maleic acid, and itaconic acid.

31. The method of claim 21, wherein said composition is not treated with UV having a wavelength of 400 nm or less, and an irradiance intensity of above 500 W/m².

32. The method according to claim 21, wherein said composition is free or essentially free of isoprenyl-alkyoxylate homo- and copolymers.

33. The method of claim 21, wherein said alkylene oxide of (a) is selected from the group consisting of ethylene oxide, propylene oxide, butylene oxide, pentene oxide, decene oxide, and dodecene oxide.

34. The method of claim 21, wherein said catalyst of (a) is selected from the group consisting of KOMe, Na-alkoxylate, Li-alkoxylate, K-alkoxylate, NaOH, and KOH.

35. The method of claim 21, wherein the reaction step (a) is performed at a temperature of 70° C. to 180° C.

36. The method according to claim 21, wherein the inert gas of (c) is N₂.

37. The method according to claim 21, wherein the color number (Gardner) is reduced from over 5.5 to below 5.

38. A composition obtainable by the method of claim 21.

39. A process for decreasing the amount of isoprene in a composition which comprises utilizing a peroxide or peroxide generating compound.

40. The process of claim 39, wherein said composition comprises an isoprenol-alkoxylate.
